(19) United States
(12) Patent Application Publication
(10) Pub. No.: US 2004/0158036 A1
(43) Pub. Date: Aug. 12, 2004

(54) Neuritogenic compound and uses thereof

(76) Inventors: Lawrence Rosenberg, Cote St. Luc (CA); Dusica Maysinger, Montreal (CA)

Correspondence Address: WOOD, PHILLIPS, KATZ, CLARK & MORTIMER, 500 W. MADISON STREET, SUITE 3800, CHICAGO, IL, 60661, US

(21) Appl. No.: 10/469,314

(22) PCT Filed: Feb. 28, 2002
(86) PCT No.: PCT/CA02/00257

(51) Int. Cl. A61K 38/10; C07K 7/08

(52) U.S. Cl. 530/326; 514/14

(57) ABSTRACT

The present invention relates to a neuritogenic compound for neurite outgrowth, which comprises the amino acid sequence: Gly Leu His Asp Pro Ser His Gly Thr Leu Pro Asn Gly Ser Gly (SEQ ID NO:3), functional derivatives and/or fragments thereof and functional peptidometics thereof. There is also provided a method for repair and/or regeneration of peripheral nervous system in a patient.

### BACKGROUND OF THE INVENTION

(a) Field of the Invention

The invention relates to a neuritogenic compound derived from a peptide fragment of Islet Neogenesis Associated Protein (INGAP) for neurite outgrowth, including for repair and/or regeneration in the peripheral nervous system.

(b) Description of Prior Art

Using a model of partial pancreatic duct obstruction, the applicants have previously demonstrated that islet cell differentiation from progenitor cells associated with the ductal epithelium could be induced in the adult pancreas (Rosenberg, L. et al. (1983) J. Surg. Res. 35, 63-72; Rosenberg, L. et al. (1990) Surgery 108, 191-197; Rosenberg, L. (1998) Microsc. Res. Tech. 43, 337-346) and that the newly formed islets functioned to reverse a diabetic state (Rosenberg, L. et al. (1988) Diabetes 37, 334-341; Watkins, P. J. (1993) Diabetes Med. 10 (Suppl. 2), 77S-78S). Evidence obtained from parabiotic studies suggested that the induction of cell proliferation and differentiation was mediated by paracrine and/or autocrine mechanisms. Using classical protein chemistry techniques, a crude soluble tissue extract was isolated from the pancreas, and was shown to stabilize or reverse streptozotocin-induced diabetes. Subsequent investigations led to the isolation of a novel gene and protein product responsible for the bioactivity of the extract. This 175 amino acid protein was termed islet-neogenesis-associated protein, INGAP.

There was reported the identification of a gene, INGAP, that shows striking homology to the pancreatitis associated protein (PAP) family of genes (7-11). The predicted protein shares the carbohydrate recognition domain (CRD) of the calcium dependent C-type lectins. INGAP plays a role in stimulation of islet neogenesis, in particular, in beta cell regeneration from ductal cells.

The cDNA sequence of a mammalian INGAP is as follows:

| | | | | |
|---|---|---|---|---|
| CTGCAAGACA | GGTACCATG | ATG CTT CCC ATG ACC CTC TGT AGG ATG TCT TGG | 52 | (SEQ ID NO: 1) |

Met Leu Pro Met Thr Leu Cys Arg Met Ser Trp

1 5 10

| | |
|---|---|
| ATG CTG CTT TCC TGC CTG ATG TTC CTT TCT TGG GTG GAA GGT GAA GAA | 100 |
| Met Leu Leu Ser Cys Leu Met Phe Leu Ser Trp Val Glu Gly Glu Glu | |
| 15 20 25 | |
| TCT CAA AAG AAA CTG CCT TCT TCA CGT ATA ACC TGT CCT CAA GGC TCT | 148 |
| Ser Gln Lys Lys Leu Pro Ser Ser Arg Ile Thr Cys Pro Gln Gly Ser | |
| 30 35 40 | |
| GTA GCC TAT GGG TCC TAT TGC TAT TCA CTG ATT TTG ATA CCA CAG ACC | 196 |
| Val Ala Tyr Gly Ser Tyr Cys Tyr Ser Leu Ile Leu Ile Pro Gln Thr | |
| 45 50 55 | |
| TGG TCT AAT GCA GAA CTA TCC TGC CAG ATG CAT TTC TCA GGA CAC CTG | 244 |
| Trp Ser Asn Ala Glu Leu Ser Cys Gln Met His Phe Ser Gly His Leu | |
| 60 65 70 75 | |
| GCA TTT CTT CTC AGT ACT GGT GAA ATT ACC TTC GTG TCC TCC CTT GTG | 292 |
| Ala Phe Leu Leu Ser Thr Gly Glu Ile Thr Phe Val Ser Ser Leu Val | |
| 80 85 90 | |
| AAG AAC AGT TTG ACG GCC TAC CAG TAC ATC TGG ATT GGA CTC CAT GAT | 340 |
| Lys Asn Ser Leu Thr Ala Tyr Gln Tyr Ile Trp Ile Gly Leu His Asp | |
| 95 100 105 | |
| CCC TCA CAT GGT ACA CTA CCC AAC GGA AGT GGA TGG AAG TGG AGC AGT | 388 |
| Pro Ser His Gly Thr Leu Pro Asn Gly Ser Gly Trp Lys Trp Ser Ser | |
| 110 115 120 | |
| TCC AAT GTG CTG ACC TTC TAT AAC TGG GAG AGG AAC CCC TCT ATT GCT | 436 |
| Ser Asn Val Leu Thr Phe Tyr Asn Trp Glu Arg Asn Pro Ser Ile Ala | |
| 125 130 135 | |
| GCT GAC CGT GGT TAT TGT GCA GTT TTG TCT CAG AAA TCA GGT TTT CAG | 484 |
| Ala Asp Arg Gly Tyr Cys Ala Val Leu Ser Gln Lys Ser Gly Phe Gln | |
| 140 145 150 155 | |
| AAG TGG AGA GAT TTT AAT TGT GAA AAT GAG CTT CCC TAT ATC TGC AAA | 532 |
| Lys Trp Arg Asp Phe Asn Cys Glu Asn Glu Leu Pro Tyr Ile Cys Lys | |
| 160 165 170 | |
| TTC AAG GTC TAGGGCAGTT CTAATTTCAA CAGAGAGCAA GCTCTGCCTA CACACCCACA | 591 |
| Phe Lys Val | |
| CCAATTCCCT TATATCATCT CTGCTGTTTT TCCTTGAAAT TATTATGAAG CTCACATGGA | 651 |
| CAAGGAAGCA AGTATGAGGA TTCACTCAGG ATATCAGTAT ATTCTGTGGT GGCTGTAACC | 711 |
| TAAAGGCTCA GAGAACAAAA ATAAAATGTC ATCAAC. | 747 |

A predicted amino acid sequence is as follows:

| Met Leu Pro Met Thr Leu Cys Arg Met Ser Trp Met Leu Leu Ser Cys | (SEQ ID NO: 2) |
|---|---|
| 1 5 10 15 | |
| Leu Met Phe Leu Ser Trp Val Glu Gly Glu Glu Ser Gln Lys Lys Leu | |
| 20 25 30 | |
| Pro Ser Ser Arg Ile Thr Cys Pro Gln Gly Ser Val Ala Tyr Gly Ser | |
| 35 40 45 | |
| Tyr Cys Tyr Ser Leu Ile Leu Ile Pro Gln Thr Trp Ser Asn Ala Glu | |
| 50 55 60 | |
| Leu Ser Cys Gln Met His Phe Ser Gly His Leu Ala Phe Leu Leu Ser | |
| 65 70 75 80 | |
| Thr Gly Glu Ile Thr Phe Val Ser Ser Leu Val Lys Asn Ser Leu Thr | |
| 85 90 95 | |
| Ala Tyr Gln Tyr Ile Trp Ile Gly Leu His Asp Pro Ser His Gly Thr | |
| 100 105 110 | |
| Leu Pro Asn Gly Ser Gly Trp Lys Trp Ser Ser Ser Asn Val Leu Thr | |
| 115 120 125 | |
| Phe Tyr Asn Trp Glu Arg Asn Pro Ser Ile Ala Ala Asp Arg Gly Tyr | |
| 130 135 140 | |
| Cys Ala Val Leu Ser Gln Lys Ser Gly Phe Gln Lys Trp Arg Asp Phe | |
| 145 150 155 160 | |
| Asn Cys Glu Asn Glu Leu Pro Tyr Ile Cys Lys Phe Lys Val. | |
| 165 170 | |

These sequences were determined from nucleic acids isolated from hamster, but it is believed that other mammalian species will contain INGAP genes which are quite similar. For example, one would expect homologous genes to contain at least about 70% identity. Closer species would be expected to have at least about 75%, 80%, or even 85% identity. In contrast, other family members of the calcium dependent C-type lectins contain at most 60% identity with INGAP. INGAP genes can be isolated from other mammals by utilizing the nucleotide sequence information provided herein.

It has been found that INGAP and fragments thereof are capable of inducing and stimulating islet cells to grow. Moreover, they are capable of inducing differentiation of pancreatic duct cells into insulin-producing β-cells. Thus many therapeutic modalities are now possible using INGAP, fragments thereof, and nucleotide sequences encoding INGAP. Therapeutically effective amounts of INGAP are supplied to patient pancreata, to isolated islet cells, and to encapsulated pancreatic islet cells, such as in a polycarbon shell.

Known conditions, which can be treated with INGAP, include diabetes mellitus, both insulin dependent and non-insulin dependent, pancreatic insufficiency, pancreatic failure, etc. Inhibition of INGAP expression can be used to treat nesidioblastosis.

However, no data have been reported on the possible effect of INGAP or INGAP peptide in the nervous system.

It would be highly desirable to be provided with a compound derived from an INGAP peptide for repair and/or regeneration of peripheral nervous system.

### SUMMARY OF THE INVENTION

One aim of the present invention is to provide a compound derived from an INGAP peptide for repair and/or regeneration of peripheral nervous system.

According to the present invention, it has now been found that a small portion of INGAP is sufficient to confer a biological activity on peripheral nervous system.

A fragment of 15 amino acids of the sequence of SEQ ID NO: 2, from amino acid 104-118 is sufficient to stimulate repair and/or regeneration of peripheral nervous system.

More precisely, such an INGAP peptide has the following amino acid sequence: Gly Leu His Asp Pro Ser His Gly Thr Leu Pro Asn Gly Ser Gly (SEQ ID NO:3).

In accordance with the present invention there is provided a neuritogenic compound, which comprises the amino acid sequence: Gly Leu His Asp Pro Ser His Gly Thr Leu Pro Asn Gly Ser Gly (SEQ ID NO:3), functional derivatives and/or fragments thereof and functional peptidometics thereof.

In accordance with the present invention there is provided use of the neuritogenic compound for enhancing neurite outgrowth (in vitro or in vivo), and more precisely such as for repair and/or regeneration of peripheral nervous system.

In accordance with the present invention there is also provided a neuritogenic pharmaceutical composition, which comprises a therapeutically effective amount of a compound of the present invention in association with a pharmaceutically acceptable carrier.

In accordance with the present invention there is provided use of the neuritogenic composition for enhancing neurite outgrowth (in vitro or in vivo), and more precisely such as for repair and/or regeneration of peripheral nervous system.

In accordance with the present invention there is also provided a method for repair and/or regeneration of peripheral nervous system in a patient, which comprises administering to said patient a therapeutically effective amount of a neuritogenic compound or of a neuritogenic composition of the present invention.

The patient may be suffering from a neuropathy, included but not limited to diabetic neuropathy, and/or a peripheral nervous system injury.

For the purpose of the present invention the following terms are defined below.

The expression “functional derivatives and/or fragments thereof and functional peptidometics thereof” is intended to mean any isomer, peptide analogues, peptide derivatives including deletion, substitution, or peptidomimetics which retain the neuroregenerative activity of the INGAP peptide has the following amino acid sequence: Gly Leu His Asp Pro Ser His Gly Thr Leu Pro Asn Gly Ser Gly (SEQ ID NO:3).

Except as otherwise expressly defined herein, the abbreviations used herein for designating the amino acids and the protective groups are based on recommendations of the IUPAC-IUB Commission on Biochemical Nomenclature (Biochemistry, 1972, 11:1726-1732).

### BRIEF DESCRIPTION OF THE DRAWINGS

FIG. 1 illustrates the effect of the INGAP peptide of the present invention on neurite outgrowth in explanted C57BL/6 dorsal root ganglia (DRG).

FIG. 2 illustrates the effects of the INGAP peptide of the present invention, 17-β-estradiol and FK506 on the long term viability of DRG explant cultures.

FIG. 3 illustrates the dose-dependent stimulation of neurite outgrowth from explanted DRG by INGAP peptide at 4 and 7 days in culture. DRG were dissected from 1-month-old C57/B16 mice and cultured in Matrigel in RPMI 1640 tissue culture media (37° C., 5% CO₂) containing 1% antibiotic in the absence or presence of drug treatments. Magnification was obtained with 4× and 10×(shown) objectives. Representative samples from three independent experiments in which the group size was n=4-5 are shown. (A) Overall time course of neurite outgrowth observed at 1, 4, and 7 days. (B) Constitutive neurite outgrowth from DRG after 7 days. (C-E) INGAP peptide treatment enhances the overall density and length of neurites in a dosedependent manner after 4 days in culture. Significant effects are observed at doses of 500 ng/mL (D) and 1000 ng/mL (E). No effects are observed at a concentration of 100 ng/mL (C). Differences from non-treated controls were considered significant where p<0.05.

FIG. 4 illustrates the effect of differentiating agents on INGAP peptide-stimulated neurite outgrowth. Explanted DRGs were cultured as described in the legend to FIG. 1 and treated with INGAP peptide (1000 ng/mL), NGF (50 ng/mL), anti-NGF antibody, or 17-(β-estradiol (10 nM). (A-C, G) INGAP peptide, NGF or 17-(β-estradiol produces strong neurite outgrowth relative to non-treated cultures (P<0.05). (E) Combination of NGF with INGAP peptide enhances neurite outgrowth in comparison, to INGAP peptide treatment alone (P<0.05). (F) Preincubation of DRG cultures with anti-NGF antibody leads to marked reduction of INGAP peptide-stimulated neurite outgrowth (P<0.05). (H) No significant enhancement of the INGAP peptide effect on neurite outgrowth is seen when applied with 17-(β-estradiol (P<0.05). Differences from non-treated controls or INGAP peptide-treated cultures were considered significant where P<0.05.

FIG. 5 illustrates the enhancement of mitochondrial activity by INGAP peptide in explanted DRG. Explanted DRG cultures were created as described in the legend to FIG. 1. After 24 h in culture, no significant effects are obtained with INGAP peptide applied in a concentration range of 100-1000 ng/mL. At 4 days in culture, a significant increase in mitochondrial activity indicated by greater MTT reduction is observed in DRGs treated with INGAP peptide at a dose of 1000 ng/mL. In longer-term cultures (7 days), INGAP peptide applied at 500 and 1000 ng/mL continue a trend toward enhanced cell viability (P>0.05). Data represent the means±SEM of three independent experiments in which the sample size was n=4-5. Differences from non-treated controls were considered significant where P<0.05.

FIG. 6 illustrates the dose-dependent induction of cell proliferation by INGAP peptide. DRG explant cultures were created as described in the legend to FIG. 1. After 4 days in culture, [³H] thymidine incorporation was assessed in DRG treated with INGAP peptide (100-1000 ng/mL) or high-dose serum (20%). High-dose INGAP peptide (1000 ng/mL) or serum produce strong increases (approximately 4.5- and 5-fold, respectively) in cell proliferation as indicated by increased [³H] thymidine incorporation. 500 ng/mL INGAP peptide treatment produces less cell proliferation after 4 days (3-fold), while low-dose INGAP peptide (100 ng/mL) has no significant effects relative to non-treated cultures. Data represent means±SEM. Differences were considered significant where P<0.05.

### DETAILED DESCRIPTION OF THE INVENTION

Islet-neogenesis-associated protein, INGAP, is a 175 amino acid pancreatic acinar protein that stimulates pancreatic duct cell proliferation in vitro and islet neogenesis in vivo. To date, the mitogenic activity of INGAP has been identified only in nonneural tissues. The present invention provides evidence that a pentadecapeptide of INGAP (INGAP peptide) acts as a mitogen in the peripheral nervous system (PNS), and that it enhances neurite outgrowth from DRGs in vitro in a time- and dose-dependent manner. The neuritogenic action of INGAP peptide correlates with an increase in [³H] thymidine incorporation (P<0.0001) and mitochondrial activity (P<0.001). Results from these studies suggest that INGAP peptide promotes Schwann cell proliferation in the DRG which releases trophic factors that promote neurite outgrowth.

A 15-amino-acid sequence (amino acids 104-118) contained within the native protein was identified as the biologically active core of INGAP. In vitro, the 175-amino-acid protein with molecular mass of approximately of 20 kDa is capable of initiating pancreatic duct cell proliferation, a process that is essential for the neogenesis of islets from precursor cells located in the ductal epithelium. Studies with streptozotocin-induced diabetic hamsters and C57BL/6J mice showed that INGAP treatment is able to ameliorate hyperglycemia in a dose-dependent manner or reverse it completely.

This 15-amino-acid region (INGAP peptide) has been manufactured by solid-phase peptide synthesis and shown to be a potent inducer of islet-cell neogenesis from cells associated with the ductal epithelium, leading to new islet formation in the normal, adult mouse, and hamster.

The present invention will be more readily understood by referring to the following examples which are given to illustrate the invention rather than to limit its scope.

### EXAMPLE 1

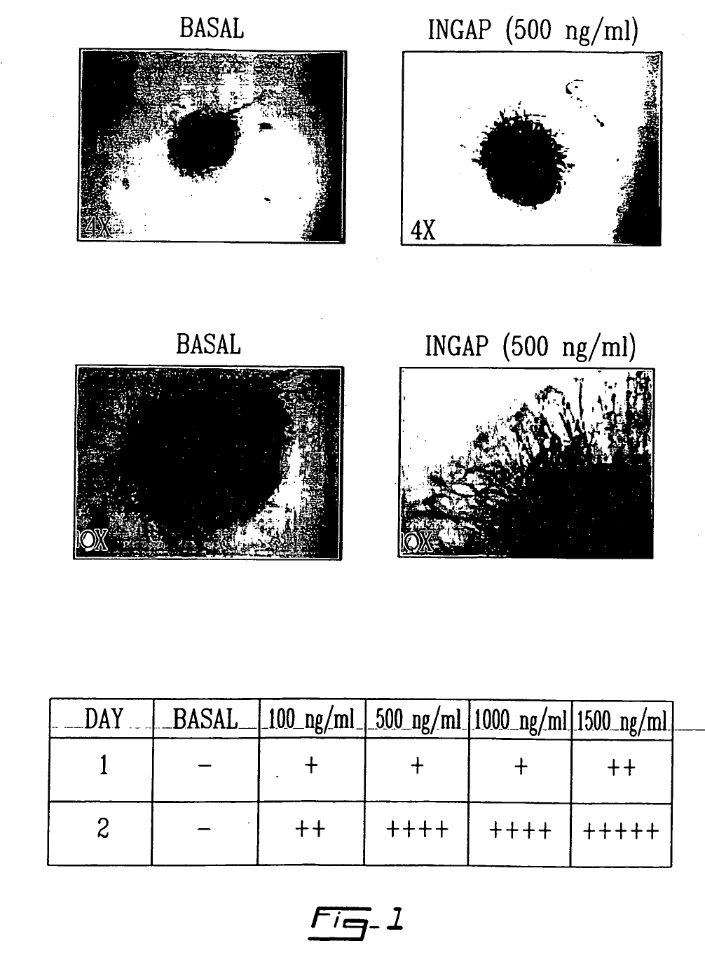

Using male C57BL/6 mice, dorsal route ganglia (DRG) explant cultures were established to examine the effect of the INGAP peptide of the present invention on neurite outgrowth (FIG. 1). DRG (L3, L4) were dissected from male C57BL/6 mice, and DRG explants were cultured in 14 uL of Matrigel in 2 ml of RPMI 1640 media+1% antibiotic.

Our studies in this in vitro model of the peripheral nervous system suggest that INGAP peptide is effective, in a dose-dependent fashion, as a neurite-promoting agent. This effect is comparable to that observed with a more conventional neurotrophic agent, such as nerve growth factor (NGF).

### EXAMPLE 2

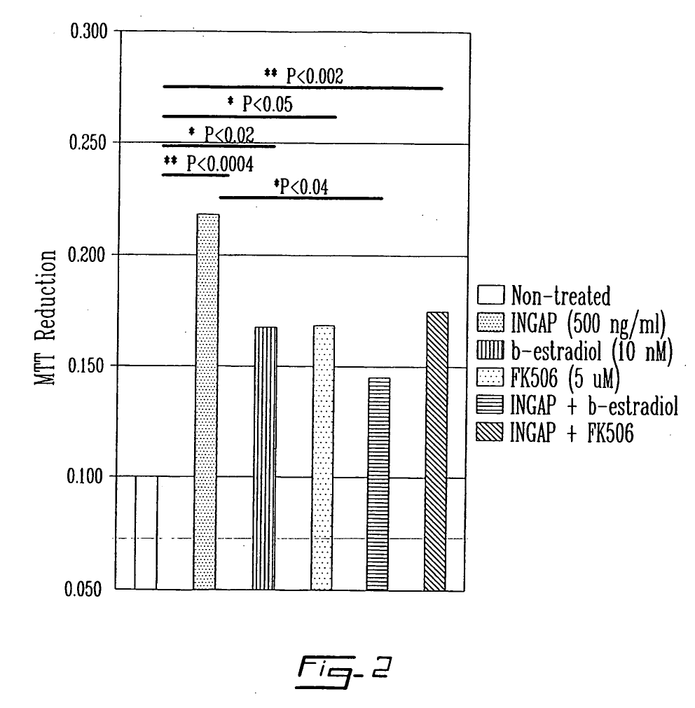

Using DRG explant cultures, we examined the effects of the INGAP peptide of the present invention, as well as other agents, on DRG cell viability using the MTT assay (FIG. 2). DRG (L3, L4) were dissected from Balb/C mice, and DRG explants were cultured in 14 uL of Matrigel in 2 ml of RPMI 1640 media+1% antibiotic. After 48 hours, INGAP, β-estradiol, and/or FK506 were added to the explant cultures as indicated. MTT assay was performed 7 days after the addition of drugs. Data were analyzed using one-way ANOVA with post-hoc Tuckey” test. Differences were consider significant where p<0.05. (n=3-4 per treatment group).

The data suggest that INGAP has a potent effect on cell viability. However, the first bar of FIG. 2 represents the viability of cells under normal conditions of culture (14 μl of Matrigel in 2 ml RPMI 1640+1% Penicillin/streptomycin). A tremendous increase in MTT reduction is seen with the addition of INGAP (doses tested were between 100 ng/mL to 1,500 ng/mL). Maximal effect was observed at 500 ng/mL and did not significantly change with 1000 Or 1,500 ng/mL (the second bar in FIG. 2 refers to 500 ng/mL of INGAP in the medium) to represent a mitogenic effect on Schwann cells. This effect would lead as well to the release of known neurotrophins, including IGF-1, IGF-2, NT-3, PDGF-B, and NGF, that could be directly responsible for inducing the neurite outgrowth demonstrated in Example 1.

This is novel information pointing to the possibility that INGAP treatment could be useful in diabetes not only because of being neogenetic but also neurotrophic.

### EXAMPLE 3

Materials and Methods

All animal work was performed according to CCAC (Canadian Council on Animal Care) guidelines, and all efforts were made to minimize animal suffering and to reduce the number of animals used.

Matrigel extracellular matrix was obtained from Becton-Dickinson U.S.A. Cell culture media (RPMI 1640 without phenol-red) was from Gibco-BRL (Life Technologies, Grand Island, N.Y.). Tissue culture plates were from Falcon (Becton-Dickinson, Franklin Lakes, N.J.). 17-R-Estradiol and (3-[4,5-dimethylthiazol-2-yl]-2,5-diphenyltetrazolium bromide) (MTT) were purchased from Sigma-Aldrich (Oakville, Ontario, Canada). NGF was obtained from Cedar Lane Laboratories (Hornsby, Ontario, Canada), and INGAP synthetic pentadecapeptide was obtained from the Sheldon protein synthesis facility at McGill University (Montreal, Quebec, Canada).

DRG (L3, L4, L5) were dissected from wild-type C57/B16 mice <, and DRG explants were cultured in 7 pL of Matrigel at 37° C. (5% CO₂) for 24 h in phenol-red free RPMI 1640 media-free supplemented with 1% penicillin/streptomycin. Neurite outgrowth was followed for 7 days and was visualized with an OLYMPUS™ BX51 system microscope equipped with a Dage-MTI CCD300-RC camera coupled to a Powermate™ 8100 Pentium™III computer (NEC Corp.) with 128 MB RAM. Bright-field microscope images were stored in uncompressed tagged image file format (TIFF) at a resolution of 600 d.p.i. and analyzed using MCID-M5+5.1 image analysis software (Imaging Research Inc.) and SYSTAT 9 statistical software (SPSS Inc.).

Drug Treatments

In the neurite outgrowth assays, INGAP (1001000 ng/mL), 17-(β-estradiol (10 nM) or NGF (50 ng/mL) and anti-NGF (1 pg/pL that completely blocks neurite outgrowth of 50 ng/mL of NGF) were added to the serum-free culture media for different times (1-7 days).

For cell viability assays, MTT was dissolved in phosphate buffered saline (1.9 mM NaH₂PO₄, 8.1 mM Na₂HPO₄, 154 mM NaCl) and applied to DRG explant cultures at a concentration of 1.2 mM for 2 h at (37° C., 5% CO₂). Media were then aspirated, and each DRG explant was incubated in 200 pL of DMSO for 1 h. 100 pL of DMSO from each explant was transferred to a 96-well plate and absorbance at 595 nm was read on a Bio-Rad Benchmark plate reader.

For [3H]thymidine incorporation assays, serum-starved DRG ex-plants cultured in Matrigel were exposed to Ingap (100-1000 ng/mL) for 48 h. 1 p Ci/ml of [3H]thymidine was added to each DRG and kept for an additional 48 h, after which time the DRG were washed four times with ice-cold trichloroacetic acid (10%). Ganglia were lysed on ice in 100pL of tissue solubilizer (NCS-II, Amersham). Lysates were transferred to individual 4 mL scintillation counting vials, and 4 mL of liquid scintillation counting cocktail (Fischer SX25-5 ScintiSafe Plus 50%) was added to each lysate. Counting was performed on a Wallac 1410 liquid scintillation counter.

In all experiments, statistical significance was determined using one-way ANOVA and post-hoc Dunnett's test. The experimental group size was n=4-5, and differences were considered significant where P<0.05.

RESULTS

Neurite outgrowth

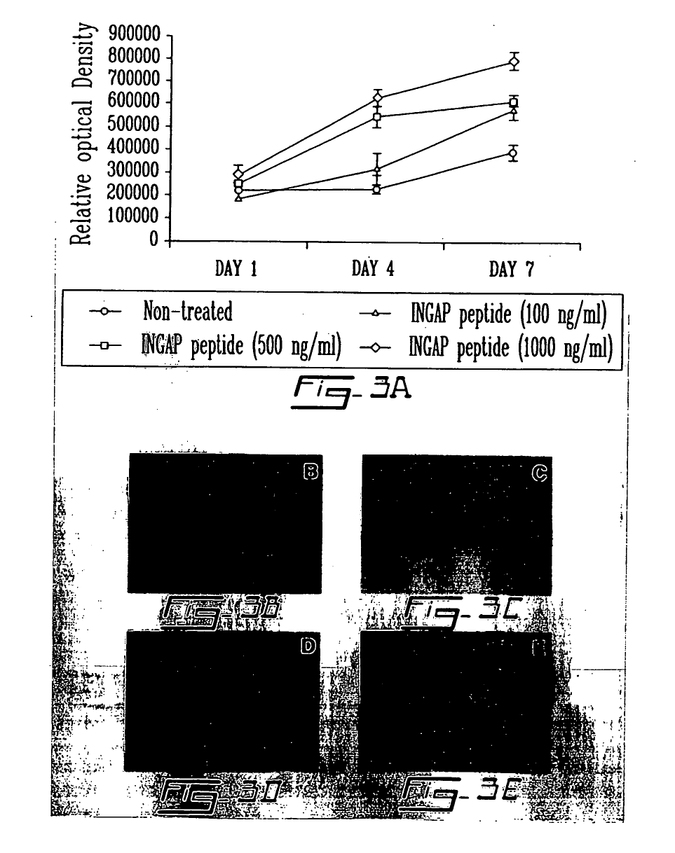

Neurite outgrowth from explanted DRG cultures was assessed at 1, 4, and 7 days in culture (FIG. 3). After 24 h, minor outgrowth was observed, with no significant differences detected between INGAP-treated DRGs and non-treated ones, regardless of the drug concentration (P>0.05, FIG. 3A). After 4 days in culture, significant enhancement of the overall length and density of neurite outgrowth was observed with INGAP treatment at concentrations of 500 ng/mL (P<0.001, FIG. 3D) and 1000 ng/mL (P<0.0001, FIG. 3E) compared to outgrowth from non-treated DRG (FIG. 3B). The lowest dose of INGAP tested, 100 ng/mL, did not have a significant effect on neurite outgrowth (P>0.05, FIG. 3C) at any time point examined. Following 7 days in culture, only INGAP applied at the concentration of 1000 ng/mL continued to significantly enhance neurite outgrowth compared to outgrowth from non-treated DRG (P<0.001, FIG. 3A).

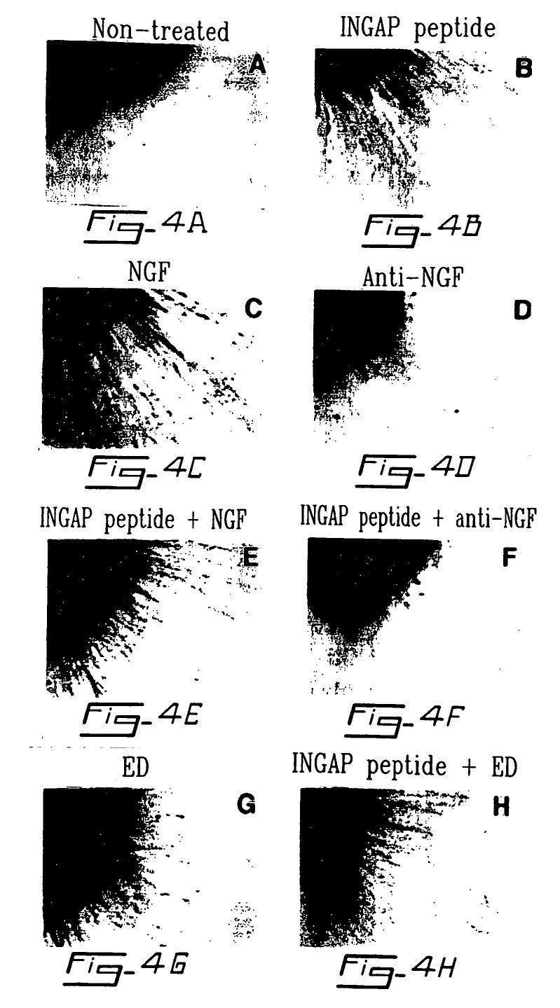

The optimal dose of INGAP (1000 ng/mL) (FIG. 4B) stimulates neurite outgrowth to an extent similar to that of NGF (50 ng/mL) (FIG. 4C, P>0.0001) or 17-β-estradiol (10 nM) (FIG. 4G, P<0.05) at both 4 days (FIG. 41) and 7 days in culture relative to non-treated controls (FIG. 4A). Enhanced effects on neurite outgrowth are observed when INGAP is applied in combination with NGF (FIG. 4E, P<0.05). In contrast, 17-(β-estradiol in combination with INGAP produces neurite outgrowth that is not significantly different from that observed with either drug alone (FIG. 4H, P<0.05). A strong reduction in INGAP-stimulated neurite outgrowth was observed in cultures pre-incubated with anti-NGF antibody (FIG. 4F, P<0.05). FIG. 4I illustrates the differential effects of the two differentiating agents on neurite outgrowth when applied alone or in combination with INGAP.

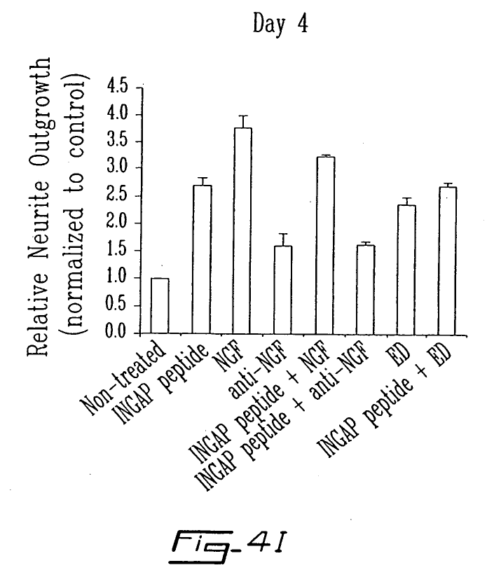

MTT assay. After 24 h in culture, the doses of INGAP tested (100, 500, 1000 ng/mL) had no significant effect on metabolically active mitochondria in explanted DRG in comparison to non-treated controls (P>0.05) (FIG. 5, Day 1). Significant differences in mitochondria activity of living cells were seen at four days in culture where INGAP at a concentration of 1000 ng/mL led to significantly larger MTT reduction (FIG. 5, Day 4, P<0.05). In longer-term cultures (7 days), INGAP at concentrations of both 500 and 1000 ng/mL showed a similar trend. (FIG. 5, Day 7, P>0.05).

[³H] Thymidine Incorporation

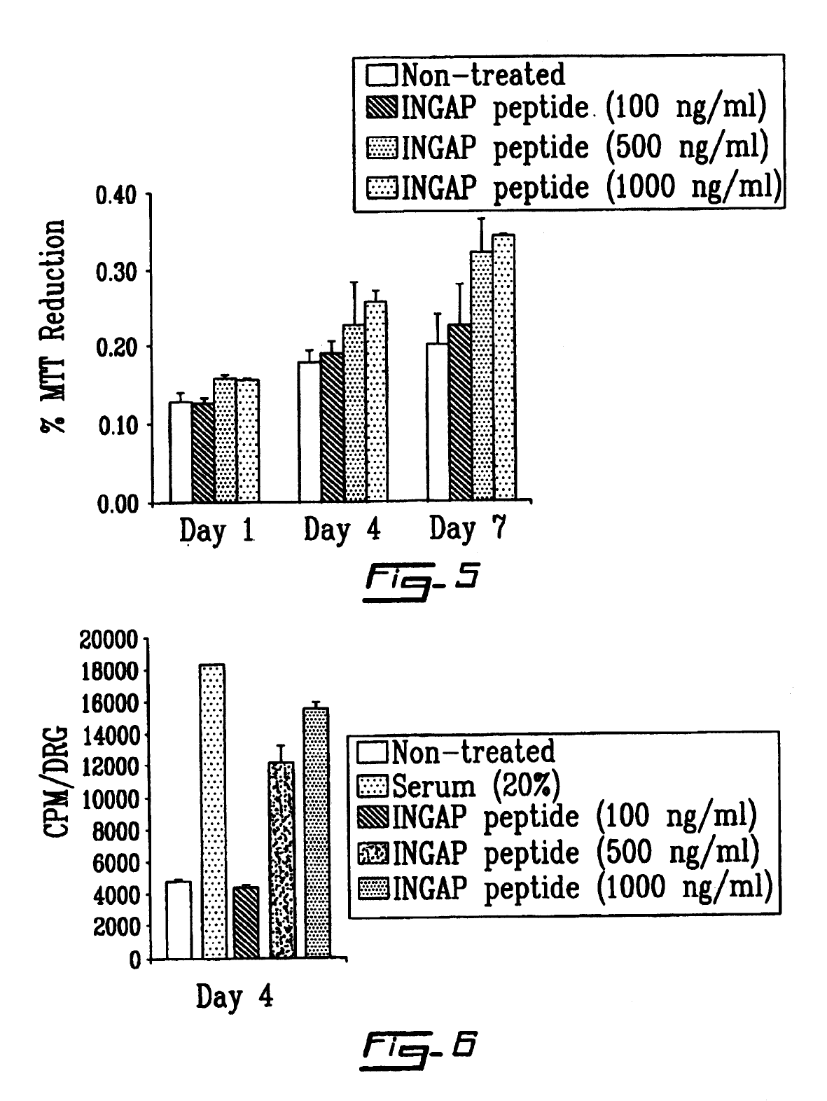

After 4 days in culture, INGAP or high-dose serum (20%) produced highly significant increases in [³H] thymidine incorporation relative to non-treated controls (FIG. 6). Serum treatment induces the greatest cellular proliferation, a 4.5-fold increase relative to non-treated controls (P<0.0001). INGAP exerts a dose-dependent effect on cell proliferation, with no significant effect observed at 100 ng/mL (P>0.05). A 3-fold increase in [³H] Thymidine incorporation at 500 ng/mL (P<0.05) and nearly 4-fold increase at 1000 ng/mL (P<0.0001) of INGAP peptide were obtained.

DISCUSSION

Results from these studies provide the first evidence that the INGAP peptide exerts a neurite-promoting effect in the peripheral nervous system. Surprisingly, and in accordance with the present invention, the applicants have found that INGAP peptide at an optimal concentration of 1000 ng/mL stimulates neurite outgrowth from the DRG explants, to an extent similar to that of the prototypical growth factor, nerve growth factor (NGF) or of phonemic steroids such as 17-(β-estradiol).

The results of the present invention demonstrate that INGAP peptide is a strong enhancer of neurite outgrowth from explanted DRG, and the DRG explant model serves well as a model of nerve axotomy. It is possible that INGAP acts directly on neurons to stimulate the nerve regeneration process, and results of MTT assays indicate that INGAP peptide treatment produces an increase in mitochondrial activity in explanted mouse DRG after 4 days in culture. It is possible that INGAP peptide also exerts an effect on the ganglionic Schwann cells. Indeed, results from thymidine incorporation assays suggest that INGAP peptide treatment leads to an increase in the number of Schwann cells in the sensory ganglia.

Among the candidate molecules that could promote neurite outgrowth under employed conditions for explanted DRGs treated with INGAP peptide, are NGF, IGFs and LIF. Indeed, our experiments utilizing anti-NGF show that at least in part, NGF is involved in promoting neurite outgrowth in the INGAP peptide-treated DRG.

In summary, we provide the first evidence that INGAP peptide exerts an effect in the peripheral nervous system, in particular, on the sensory ganglia. We conclude that INGAP peptide enhances neurite outgrowth from explanted DRG cultures by inducing the proliferation of Schwann cells, which may then produce the growth factors required to support nerve regeneration. These findings are of particular relevance to the peripheral neuropathy associated with almost half of the cases of diabetes, in that the INGAP may represent a potential therapeutic that not only restores normoglycemia via islet neogenesis but also ameliorates one of the most common and devastating complications of the disease.

While the invention has been described in connection with specific embodiments thereof, it will be understood that it is capable of further modifications and this application is intended to cover any variations, uses, or adaptations of the invention, following, in general, the principles of the invention and including such departures from the present disclosure as come within known or customary practice within the art to which the invention pertains and as may be applied to the essential features hereinbefore set forth, and as follows in the scope of the appended claims.

What is claimed is:

1. A neuritogenic compound which comprises the amino acid sequence: Gly Leu His Asp Pro Ser His Gly Thr Leu Pro Asn Gly Ser Gly (SEQ ID NO:3), functional derivatives and/or fragments thereof and functional peptidometics thereof.

2. Use of the compound of claim 1 for enhancing neurite outgrowth.

3. The use of claim 2, wherein said enhancing of neurite outgrowth is for repair and/or regeneration of peripheral nervous system in a patient

4. A neuritogenic pharmaceutical composition, which comprises a therapeutically effective amount of a compound claim 1 in association with a pharmaceutically acceptable carrier.

5. Use of the composition of claim 4 for enhancing neurite outgrowth.

6. The use of claim 5, wherein said enhancing of neurite outgrowth is for repair and/or regeneration of peripheral nervous system in a patient

7. A method for repair and/or regeneration of peripheral nervous system in a patient, which comprises administering to said patient a therapeutically effective amount of a compound claim 1 or of a composition of claim 4.

8. The method of claim 7, wherein said patient is suffering from a neuropathy and/or a peripheral nervous system injury.

9. The method of claim 8, wherein said neuropathy is diabetic neuropathy.


SEQUENCE LISTING (3 sequences)

SEQ ID NO: 1 - DNA, 747 bp, Artificial sequence
  CDS (20)...(541)
  Islet Neogenesis Associated Protein (INGAP)
ctgcaagacaggtaccatgatgcttcccatgaccctctgtaggatgtcttggatgctgctttcctgcctgatgttcctttcttgggtggaaggtgaagaatctcaaaagaaactgccttcttcacgtataacctgtcctcaaggctctgtagcctatgggtcctattgctattcactgattttgataccacagacctggtctaatgcagaactatcctgccagatgcatttctcaggacacctggcatttcttctcagtactggtgaaattaccttcgtgtcctcccttgtgaagaacagtttgacggcctaccagtacatctggattggactccatgatccctcacatggtacactacccaacggaagtggatggaagtggagcagttccaatgtgctgaccttctataactgggagaggaacccctctattgctgctgaccgtggttattgtgcagttttgtctcagaaatcaggttttcagaagtggagagattttaattgtgaaaatgagcttccctatatctgcaaattcaaggtctagggcagttctaatttcaacagagagcaagctctgcctacacacccacaccaattcccttatatcatctctgctgtttttccttgaaattattatgaagctcacatggacaaggaagcaagtatgaggattcactcaggatatcagtatattctgtggtggctgtaacctaaaggctcagagaacaaaaataaaatgtcatcaac

SEQ ID NO: 2 - protein, 174 aa, Artificial sequence
  Islet Neogenesis Associated protein (INGAP)
MLPMTLCRMSWMLLSCLMFLSWVEGEESQKKLPSSRITCPQGSVAYGSYCYSLILIPQTWSNAELSCQMHFSGHLAFLLSTGEITFVSSLVKNSLTAYQYIWIGLHDPSHGTLPNGSGWKWSSSNVLTFYNWERNPSIAADRGYCAVLSQKSGFQKWRDFNCENELPYICKFKV

SEQ ID NO: 3 - protein, 15 aa, Artificial Sequence
  INGAP peptide
GLHDPSHGTLPNGSG